SEQUENCE LISTING (8 sequences)

SEQ ID NO: 1 - DNA, 2931 bp, Homo sapiens
  CDS (1)..(2928)
  misc_structure (1668)..(1670): codon 560 that can mutate to encode Glycine
atgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctga

SEQ ID NO: 2 - protein, 976 aa, Homo sapiens [sequence omitted]

SEQ ID NO: 3 - DNA, 1947 bp, Homo sapiens
  CDS (1)..(1944)
  misc_structure (1081)..(1083): codon 361 that can mutate to encode Serine
atggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccacgctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctag

SEQ ID NO: 4 - protein, 648 aa, Homo sapiens [sequence omitted]

SEQ ID NO: 5 - DNA, 3450 bp, Homo sapiens
  CDS (1)..(3447)
  misc_structure (730)..(765): encode amino acids 244-255 for forming the sides of the enzymatic pocket
atggggcagcagcctggaaaagtacttggggaccaaagaaggccaagcttgcctgccctgcattttatcaaaggagcagggaagaaggaatcatcgaggcatgggggtccacactgcaatgtttttgtggaacatgaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccggcggccactcaggacttcagcaagctcctcagttcggtgaaggaaatcagtgacatagtgcagaggtag

SEQ ID NO: 6 - protein, 1149 aa, Homo sapiens
MGQQPGKVLGDQRRPSLPALHFIKGAGKKESSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 7 - DNA, 2982 bp, Homo sapiens
  CDS (1)..(2979)
  misc_structure (2053)..(2058): codons 835 and 836 that encode amino acids to help forming the enzymatic site
atgccggcgttggcgcgcgacgcgggcaccgtgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaagctgccgctgtggaagtggatgtatctgcttccatcacactgcaagtgctggtcgatgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggacggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtaggaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtag

SEQ ID NO: 8 - protein, 993 aa, Homo sapiens
MPALARDAGTVPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVGKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

(19) United States
(12) Patent Application Publication
(10) Pub. No.: US 2004/0005623 A1
(43) Pub. Date: Jan. 8, 2004

(54) Method of determining tumor sensitivities to therapeutic drugs

(76) Inventor: B. Jack Longley JR., Hamden, CT (US)

Correspondence Address: QUARLES & BRADY LLP, 411 E. WISCONSIN AVENUE, SUITE 2040, MILWAUKEE, WI, 53202-4497, US

(21) Appl. No.: 10/457,954

(22) Filed: Jun. 10, 2003

Related U.S. Application Data
(60) Provisional application No. 60/387,406, filed on Jun. 10, 2002.
(60) Provisional application No. 60/387,370, filed on Jun. 10, 2002.

(51) Int. Cl. C12Q 1/68; G01N 33/574; G06F 19/00; G01N 33/48; G01N 33/50

(52) U.S. Cl. 435/6; 435/7.23; 702/19

(57) ABSTRACT

The present invention provides a new method for predicting whether or not a tumor (cancer, sarcoma, melanoma, etc.) in a patient will respond to a specific drug known to inhibit an enzyme that contributes to the pathogenesis of that type of tumor. The method involves conducting a comparative analysis between the enzymatic site of a reference enzyme known to interact with the drug and the enzymatic site of a target enzyme produced by the tumor cells of the patient. Identification of differences between the alleles of the two enzymes thus provides a basis for determining whether or not the drug will interact with the target enzyme in the tumor cells of the patient so as to inhibit its contribution to the pathogenesis of the tumor.

### CROSS-REFERENCE TO RELATED APPLICATION

This application claims priority to U.S. Provisional Patent Applications Nos. 60/387,406 and 60/387,370, both filed Jun. 10, 2002.

### STATEMENT REGARDING FEDERALLY SPONSORED RESEARCH OR DEVELOPMENT

This invention was developed in part using United States Government support awarded by the National Institute of Health under grant number RO1-AR43356. The United States Government has certain rights in this invention.

### BACKGROUND OF THE INVENTION

The selection of drugs for use in the treatment of individual cancer patients is based largely on results obtained in previous clinical trials and physician experience. Unfortunately, a physician's experience and prior clinical data may not accurately reflect how a cancer patient will respond to a particular drug application.

Recent studies have attempted to develop in vitro drug sensitivity systems, commonly referred to as chemosensitivity tests, to predict whether a specific anti-cancer drug will be effective in fighting cancer in an individual patient. Such systems take advantage of recent advances in cancer research which now allow cancer cells from a patient to be grown in test tubes and exposed to various anti-cancer medications. The observed response of the cancer cells to the applied drugs may then provide insight as to whether a particular drug application will effectively treat the cancer.

Two approaches, the cell sensitivity assay and the human tumor colony-forming assay, have shown some promise but are not yet practical for general application. In the human tumor colony-forming assay, the patient's cancer cells are placed in test tubes where they are exposed to the anti-cancer drugs being screened. After a short period of exposure, the exposed cells are then placed in a growth medium to determine if they will grow. Little or no growth indicates that the applied drug has been effective and may serve as a likely candidate for treating the cancer in that patient. The cell sensitivity assay, on the other hand, grows the cells first and then tests them for their drug sensitivity.

Although both studies have shown some value in predicting drug effectiveness, each test continues to have several disadvantages that limit their routine use. For instance, cells from certain cancers, such as colon, breast and lung cancers, are hard to grow in vitro such that an application of either test is unlikely. In addition, both tests often require between 2 to 3 weeks to provide results as the cells require time to grow and proliferate. The tests are also not applicable to pharmaceuticals that require interaction with bodily chemicals not present in the in vitro setting, nor is it yet possible to accurately duplicate in the in vitro environment the drug concentrations and duration of exposure of the cancer cells to the drugs within the body. As a result, the cell sensitivity assay and the human tumor colony-forming assay are very rarely used.

What is needed is a new technique that can more rapidly and accurately predict whether or not a specific anti-cancer drug is likely to be effective in treating cancer. For example, the KIT protein, which is the tyrosine kinase receptor for stem cell factor (SCF) is encoded by the c-KIT proto-oncogene. KIT is essential for normal development of mast cells and certain types of gastrointestinal stromal cells in humans and other mammals. Mutations of the c-KIT gene, however, are responsible for various types of tumors, including Gastrointestinal Stromal Tumors (GISTs) believed to originate from different c-KIT mutations that cause spontaneous activation of the KIT tyrosine kinase. Sporadic Adult Human Mastocytosis (SAHM) is also caused by specific mutations in c-KIT codon 816, which constitutively activate the KIT kinase. Certain mast cell lines and canine mast cell tumors also express a variety of other activating c-KIT mutations.

Studies have shown that small molecules that inhibit mutant activated KIT effectively kill these cell lines and may be clinically effective against GISTs. Unfortunately, recent studies have shown that drugs known to be effective in treating various forms of GISTs and some mast cell tumor lines will not be effective in treating SAHM because of resistance of the particular SAHM mutant KIT protein to these same drugs. What is needed is a new technique that can more rapidly and accurately predict whether or not a specific drug known to inhibit one form of the tumor expressing activated KIT protein will also be able to effectively interact with and treat other tumors, such as Sporadic Adult Human Mastocytosis arising in individual patients.

### BRIEF SUMMARY OF THE INVENTION

The present invention is summarized as a new method of predicting whether or not a tumor will respond to a specific drug which is known to inhibit an enzyme that contributes to the pathogenesis of that type of tumor. The method involves conducting a comparative analysis between the enzymatic site of a reference enzyme known to interact with the drug and the enzymatic site of the target enzyme produced by the tumor cells of the patient. Identification of differences between the alleles of the two enzymes thus provides a basis for determining whether or not the drug will interact with the target enzyme in the tumor cells of the patient so as to inhibit its contribution to the pathogenesis of the tumor.

The method includes the steps of obtaining a sample of tumor cells from a patient, determining the allele of the enzymatic site of the target enzyme to be tested for its ability to interact with a drug of interest, and comparing the allele of the target enzyme to a reference providing information regarding the allele of a reference enzyme known to interact with the drug of interest. The allele of the enzymatic site of the target enzyme is determined by analyzing a biomolecule from the tumor cells. The biomolecule may be either a nucleotide sequence encoding an enzymatic site of the target enzyme, a nucleotide sequence encoding a fragment of the enzymatic site of the target enzyme, a polypeptide fragment from the enzymatic site of the target enzyme, or the enzyme itself. The analysis of the biomolecule may be conducted using either a gene chip, a probe, monoclonal or polyclonal antibodies, or by sequencing the biomolecule or performing a nucleotide polymorphism analysis of the biomolecule.

To determine if the target enzyme is likely to interact or not interact with the drug of interest, the allele of the target enzyme is compared to a reference providing information regarding the allele of an enzymatic site of a reference enzyme known to interact with the drug of interest. The reference may be either a reference biomolecule, a reference database, or a control. The reference biomolecule may include either a polynucleotide encoding the enzymatic site of the reference enzyme, or a polynucleotide encoding a fragment of the enzymatic site of the reference enzyme, including a labeled or unlabelled probe or a gene chip designed to include polynucleotide fragments representing portions of the nucleotide sequence encoding the enzymatic site of the reference enzyme, or polypeptide fragments from the enzymatic site of the reference enzyme, the reference enzyme itself, or monoclonal or polyclonal antibodies having an antigen-binding region located within the enzymatic site of the reference enzyme. The reference database will generally include a selection of data containing information regarding the enzymatic site of the reference enzyme.

A finding that a target enzyme is likely to interact or to not interact with a drug of interest may be further enhanced when combined with evidence of expression and/or activation of the target enzyme using any one of a number of additional studies. An example of one such study may include the detection of specific enzyme activation by identification of the phosphorylation state of the target enzyme. A second additional study may include determining tumor drug sensitivity by direct testing the sensitivity of the target enzyme to specific drugs in vitro.

The present invention also includes a method for predicting whether or not a tumor expressing activated KIT will respond to a specific drug which is known to inhibit other KIT positive tumors. The method involves conducting a comparative analysis as described above between the enzymatic site of a c-KIT reference enzyme known to interact with the drug and the enzymatic site of the c-KIT enzyme produced by the tumor cells of the patient, referred to as the c-KIT target enzyme. Identification of differences between the alleles of the two enzymes thus provides a basis for determining whether or not the drug will effectively interact with the c-KIT target enzyme in the patient's tumor cells.

The present invention also includes a method for predicting whether or not a tumor expressing activated MEK will respond to a specific drug which is known to inhibit other MEK positive tumors. The method involves conducting a comparative analysis as described above between the enzymatic site of a Raf-1 reference enzyme known to interact with the drug and the enzymatic site of the Raf-1 enzyme produced by the tumor cells of the patient. Identification of differences between the alleles of the two enzymes thus provides a basis for determining whether or not the drug will effectively interact with the Raf-1 target enzyme in the patient's tumor cells.

The present invention also includes a method for predicting whether or not a tumor expressing the bcr-abl oncogene will respond to a specific drug which is known to inhibit other BCR-ABL positive tumors. The method involves conducting a comparative analysis as described above between the enzymatic site of a BCR-ABL reference enzyme known to interact with the drug and the enzymatic site of the BCR-ABL enzyme produced by the tumor cells of the patient. Identification of differences between the alleles of the two enzymes thus provides a basis for determining whether or not the drug will effectively interact with the BCR-ABL target enzyme in the patient's tumor cells.

The present invention also includes a method for predicting whether or not a tumor expressing the flt3 oncogene will respond to a specific drug which is known to inhibit other FLT3 positive tumors. The method involves conducting a comparative analysis as described above between the enzymatic site of a FLT3 reference enzyme known to interact with the drug and the enzymatic site of the FLT3 enzyme produced by the tumor cells of the patient. Identification of differences between the alleles of the two enzymes thus provides a basis for determining whether or not the drug will effectively interact with the FLT3 target enzyme in the patient's tumor cells.

The present invention also encompasses kits for carrying out the method of the present invention. The kit will generally comprise a research tool for use in determining the allele of the target enzyme and a reference for comparing the allele of the target enzyme against a reference enzyme known to interact with one or more drugs of interest. The research tool will generally include materials for isolating and/or analyzing the biomolecule from the tumor of the patient, such as a gene chip, labeled or unlabeled probes, monoclonal or polyclonal antibodies, or materials for sequencing the biomolecule or identifying nucleotide polymorphisms. The included reference will vary depending upon the type of tumor and/or drug under investigation, and will generally include either a reference biomolecule, a reference database or a control as described above.

One advantage of the present invention is that it increases the efficiency of in vitro testing because it does not require the demonstration of specific regulatory type mutations or events, which may be more difficult to demonstrate than enzymatic site type mutations. A second advantage is that it is also useful in cases of ligand induced activation, such as autocrine activation, since identification of this form of enzymatic activation may also be difficult. A third advantage is that it does not require in vitro culture of the patient's tumor cells with the associated complexity, expense and potential for bias. Other advantages will be readily discernable upon review of the detailed description and examples set forth below.

### BRIEF DESCRIPTION OF THE SEVERAL VIEWS OF THE DRAWINGS

FIG. 1 illustrates differential sensitivity of “regulatory type” and “enzymatic pocket” type, and differentially substituted codon 816 mutant KIT to KIT inhibitors.

FIG. 2 illustrates differential sensitivity of V560G and D816V mutant KIT in HMC 1 subclones to KIT inhibitors.

FIG. 3 illustrates the induction of apoptosis in HMC1.1 but not in HMC1.2 cells by KIT inhibitors.

FIG. 4 illustrates differential effects of KIT inhibitors on the growth of HMC1.1 and HMC 1.2 cells.

### DETAILED DESCRIPTION OF THE INVENTION

The present invention is a new method of predicting whether or not a tumor (cancer, sarcoma, melanoma, etc.) in a patient will respond to a specific drug known to inhibit similar tumors. The method involves conducting a comparative analysis between the enzymatic site of a reference enzyme known to interact with the drug and the enzymatic site of an analogous enzyme produced by the tumor cells of the patient. Identification of differences between the alleles of the two enzymes thus provides a basis for determining whether or not the drug will interact with the target enzyme in the tumor cells of the patient so as to inhibit its contribution to the pathogenesis of the tumor. The present invention also includes a method for predicting whether or not a tumor expressing activated KIT will respond to a specific drug which is known to inhibit other KIT positive tumors, as well as kits for practicing the present invention.

Mutations causing ligand independent constitutive phosphorylation and activation of certain enzymes have been shown to transform cell lines from factor-dependent growth to factor-independent growth in vitro and indolent tumors to aggressive tumors in vivo. It is recognized that different types of mutations can activate these enzymes through two different types of mechanisms. One type, called “enzymatic pocket” type mutations, includes mutations which affect the structure of the enzymatic site of the enzyme. For example, one such mutation may include the D816V substitution in KIT kinase. The D816V substitution is characteristic of adult human mastocytosis and affects the activation loop at the entrance to the enzymatic “pocket” formed by the split intracellular domain of KIT kinase. The second type, termed “regulatory type” mutations, does not affect the amino acids which directly form the enzymatic site, but instead affect portions of the molecule which regulate enzyme activity. For instance, an amphipathic alpha helix discovered in the intracellular juxtamembrane region of KIT suppresses phosphorylation and kinase activity in ligand unoccupied KIT, thereby regulating KIT activity. Mutations which disrupt this helix cause a release of inhibitory regulation, thus causing constitutive activation of KIT. Other potential mechanisms for regulatory type mutations could include effects on substrate access to the enzymatic site, effects on the binding of signal transducing or regulatory molecules to the enzyme polypeptide, or the induction of ligand independent dimerization with subsequent autophosphorylation and activation.

The distinction between regulatory and enzymatic pocket type mutations has therapeutic implications. Data now shows that molecules (i.e., therapeutic drugs) which bind to and obstruct the function of the wild type enzymatic site will not only effectively inhibit ligand-induced activation but may also inhibit activation by mutations affecting intra-molecular regulation of kinase activity (regulatory type mutations). However, the same drug may not bind at all to the altered enzymatic site of an enzyme bearing an enzymatic pocket mutation. Conversely, inhibitors which bind to an altered enzymatic pocket might not bind effectively to or inhibit the wild type enzymatic site. Thus, a kinase inhibitor which binds to the wild type enzymatic pocket would be predicted to also block activation by juxtamembrane regulatory region type activating mutations and could therefore be used to treat tumors expressing such mutations, but might not be effective at treating tumors expressing activation loop mutations.

The present invention provides a method for determining whether a drug of interest will interact with a target enzyme in the tumor cells of a patient. The method is performed by first obtaining a sample of tumor cells from the patient. The manner in which the tumor cells are collected will depend primarily on the type of tumor under consideration. For example, tumor cells from various types of lymphomas, carcinomas and sarcomas may be obtained by conducting a biopsy of the tumor itself, while tumor cells from various types of leukemia may be obtained by collecting a sample of the patient's blood or bone marrow. In the case of familial tumors caused by inherited polymorphisms, non-tumor DNA such as DNA extracted from buccal mucosal epithelial cells or peripheral blood lymphocytes may be used.

The tumor cells are analyzed to determine the allele of the enzymatic site of the target enzyme. As used herein, the allele of the enzymatic site of the target enzyme refers to the physical characteristics of the enzymatic site arising from the gene encoding the enzyme. The allele of the enzymatic site may be determined using any technique known in the art for identifying the physical characteristics of gene segments and functional proteins. In general, the allele will be determined by analyzing a biomolecule associated with the enzymatic site of the target enzyme. The biomolecule will typically include either a nucleotide sequence of RNA or DNA that encodes at least a portion of the enzymatic site of the enzyme, or a polypeptide fragment from the enzymatic site of the target enzyme, or the target enzyme itself.

The present invention also provides a method for determining whether a drug of interest will interact with a c-KIT target enzyme in the tumor cells of a patient. The method is performed by first obtaining a sample of tumor cells from the patient. The tumor cells are then analyzed to determine the allele of the enzymatic site of the c-KIT target enzyme. As used herein, the allele of the enzymatic site of the c-KIT target enzyme refers to the physical characteristics of the enzymatic site arising from the gene encoding the c-KIT target enzyme. The allele of the c-KIT target enzyme may be determined using any technique known in the art for identifying the physical characteristics of gene segments and functional proteins. In general, the allele will be determined by analyzing a c-KIT biomolecule associated with the enzymatic site of the c-KIT target enzyme. The c-KIT biomolecule will typically include either a nucleotide sequence of RNA or DNA that encodes at least a portion of the enzymatic site of the c-KIT target enzyme, or a polypeptide fragment from the enzymatic site of the c-KIT target enzyme, or the c-KIT target enzyme itself.

In one embodiment, the tumor cells are processed in order to analyze the enzymatic site of the target enzyme by determining the amino acid structure forming the target enzyme's enzymatic site. The amino acid structure may be deduced using any one of the many techniques known in the art for deducing amino acid structures, including without limitation the use of monoclonal antibodies, polyclonal antibodies, or by sequencing the isolated polypeptide or the underlying nucleotide sequence encoding the polypeptide.

For example, monoclonal antibodies may be produced having an antigen-binding region specific to a binding region within the enzymatic site of the target enzyme or a c-KIT reference enzyme as discussed below. Monoclonal antibodies can be produced using well-established hybridoma techniques first introduced by Kohler and Milstein (see, Kohler and Milstein, “Continuous Cultures of Fused Cells Secreting Antibody of Pre-Defined Specificity”, Nature, 256:495-97 (1975)). These techniques involve the injection of an immunogen (e.g., cells or cellular extracts carrying the antigen or purified antigen) into an animal (e.g., mouse) so as to elicit a desired immune response in that animal. After a sufficient time, antibody-producing lymphocytes are obtained from the animal either from the spleen, lymph nodes or peripheral blood. Preferably, lymphocytes are obtained from the spleen. The splenic lymphocytes are then fused with a myeloma cell line, usually in the presence of a fusing agents such as polyethylene glycol (PEG). Any number of myeloma cell lines may be used as a fusion partner according to standard techniques. For example, one such myeloma cell line may include Sp2/0-Ag14 myeloma, non-secreting, mouse cell line (ATCC CRL 1581).

The resulting cells, which include the desired hybridomas, are then grown in a selective medium, such as HAT medium. In this medium, only successfully fused hybridoma cells survive while unfused parental myeloma or lymphocyte cells die. The surviving cells are then grown under limiting conditions to obtain isolated clones and their supernatants screened for the presence of antibodies having a desired specificity. Positive clones may then be subcloned under limiting dilution conditions and the desired monoclonal antibodies isolated. Hybridomas produced according to these methods can be propagated in vitro or in vivo (in ascites fluid) and purified using common techniques known in the art. Methods for purifying monoclonal antibodies include ammonium sulfate precipitation, ion exchange chromatography, and affinity chromatography (see, e.g., Zola et al., “Techniques for the Production and Characterization of Monoclonal Hybridoma Antibodies”, in Monoclonal Hybridoma Antibodies: Techniques and Applications, pp. 51-52 (Hurell, ed., CRC Press, 1982)).

Once purified monoclonal antibodies are obtained, epitope mapping may be performed to determine which peptide segment (or antigen-binding region) of the target enzyme is recognized by each particular antibody. The purpose for the epitope mapping is to have a well characterized monoclonal antibody. Ideally, monoclonal antibodies with different specificity to the same target enzyme can be prepared so that researchers have probes for different parts of the target enzyme under investigation. Monoclonal antibodies may also be prepared to provide probes that mimic the binding affinity of various drugs under investigation.

In a second embodiment, the tumor cells are processed in order to obtain at least a portion of the nucleotide sequence of the enzymatic site of the target enzyme. The nucleotide sequence will be either RNA or DNA (including directly isolated, copied or genomic forms) and may include either polynucleotides encoding a portion of the enzymatic site, polynucleotides encoding the entire enzymatic site, or polynucleotides encoding the entire target enzyme. Such polynucleotides may be either isolated and purified from the other biological materials derived from the sample of tumor cells, or maintained in combination with the other biological materials.

The manner in which the polynucleotides are maintained will depend primarily upon the techniques used to deduce their nucleotide sequence to determine the allele of the enzymatic site for which they encode. The polynucleotides may be analyzed using any one of the many techniques known in the art for discerning the nucleotide sequence of polynucleotide material. For example, the polynucleotide may be subjected to basic sequencing techniques as are well known in the art. The polynucleotide may also be placed in direct contact with labeled or unlabelled polynucleotide probes designed to deduce the sequence of the polynucleotide or to identify the presence of specific nucleotide sequences. The polynucleotide may also be subjected to nuclear polymorphism analysis using technology such as the Invader technology presently provided by Third Wave Technology, Inc. The polynucleotide may also be placed in contact with a gene chip (microarray) including polynucleotide fragments designed to deduce the sequence of or presence of specific nucleotide sequences. Gene chips and labeled probes may also be used as a platform for performing a direct comparison of the target enzyme's enzymatic site with the enzymatic site of a reference biomolecule known to interact with the drug of interest.

To determine whether or not a drug of interest will interact with the enzymatic site of the target enzyme in the tumor of the patient, the allele of the enzymatic site of the target enzyme must be compared to a reference providing information regarding the allele of the enzymatic site of a reference enzyme known to interact with the drug of interest. The reference may include either a reference biomolecule, a reference data base or a control, depending upon the techniques utilized in practicing the present invention.

The reference biomolecule will generally include a biological molecule representing the whole or a portion of the physical structure of the enzymatic site of the reference enzyme. For example, the reference biomolecule may include a polynucleotide encoding the enzymatic site of the reference enzyme, or a polynucleotide encoding a fragment of the enzymatic site of the reference enzyme, including a labeled or unlabelled probe or a gene chip designed to include polynucleotide fragments representing portions of the nucleotide sequence encoding the enzymatic site of the reference enzyme. The reference biomolecule may also include polypeptide fragments from the enzymatic site of the reference enzyme, the reference enzyme itself, or monoclonal or polyclonal antibodies having an antigen-binding region located within the enzymatic site of the reference enzyme.

The reference database will generally include a selection of data containing information regarding the allele of the enzymatic site of the reference enzyme. For example, the database may include a selection of alleles known to interact, or to not interact, with the drug of interest. The database may also include a listing of several drugs and the alleles for which they have shown the ability to interact or to not interact. The database may also include a listing of the nucleotide sequence or amino acid sequence of those enzymatic sites known to interact, or to not interact, with one or more types of anti-cancer drugs. The database may also include a listing of specific probes and antibodies associated with enzymatic sites known to interact, or to not interact, with one or more such anti-cancer drugs.

The allele of the enzymatic site of the target enzyme is compared to the reference in order to determine if there exists mutations causing amino acid substitutions, deletions, insertions or inversions at the enzymatic site which cause a change in the ability of the enzymatic site to interact with the drug of interest. For example, the allele of the enzymatic site of the target enzyme could be analyzed to determine the existence of a mutation that causes an amino acid substitution which results in a change in the size or polarity of a segment of the enzyme, or a change in the nature of the side chains of the amino acid(s) forming the enzymatic site. Such an analysis may be performed by simply comparing the nucleotide sequence or amino acid sequence of the enzymatic site of the target enzyme with the nucleotide sequence or amino acid sequence of the enzymatic site of the reference enzyme. The analysis may also be performed using either probes, or monoclonal or polyclonal antibodies, or nucleotide polymorphism techniques as described above. It is also envisioned that other techniques not described herein but well known to those skilled in the art may also be employed to detect differences between the enzymatic sites of the target enzyme and the reference enzyme.

A finding that a target enzyme is likely to interact or to not interact with a drug of interest may be further enhanced when combined with evidence of expression and/or activation of the target enzyme, using any one of a number of additional studies. An example of one such study may include the detection of specific enzyme activation by identification of the phosphorylation state of the target enzyme. This can be done either in situ on sections of tumor by histochemical or other means, or in vitro, for instance by western blotting or enzyme or ligand/receptor linked immunologic analysis (ELISA) of target enzyme extracted from the tumor.

A second additional study may also include determining tumor drug sensitivity by direct testing the sensitivity of the target enzyme to specific drugs in vitro. This may be done, for instance, by expression of the target enzyme by transient transfection and determination of the drug sensitivity of the expressed enzyme by western blotting. Because it is more labor intensive and therefore more expensive, this additional component would normally only be used if the target enzyme was shown to be mutated in a way that directly altered its enzymatic site (i.e., if the first tests(s) in this approach showed that the enzymatic site of the target enzyme differed from that of the reference enzyme).

The present invention differs from previous approaches to determining tumor sensitivity by its unique focus on the primary sequence of the enzymatic site, and depends on the classification system of regulatory type mutations and enzymatic site (enzymatic pocket) mutations as described by Longley et al., “Classes of c-KIT Activating Mutations: Proposed Mechanisms of Action and Implications for Disease Classification and Therapy”, Leukemia Research, (Vienna Mastocytosis Conference, July 2001). This classification system states that oncongenic enzymes that are activated by mutations which affect the enzymatic site of the molecule are more likely to show different drug inhibition profiles than enzymes that are activated by their ligands or by regulatory type mutations/events which do not affect the primary structure of the enzymatic site.

As a result, the present invention increases the efficiency of in vitro testing because it does not require the demonstration of specific regulatory type mutations or events, which may be more difficult to demonstrate than enzymatic site type mutations. It is also useful in cases of ligand induced activation such as autocrine activation, since identification of this form of enzymatic activation may also be difficult.

To facilitate in vitro testing, the present invention also encompasses kits for carrying out the method of the present invention. The kit will generally comprise a research tool for use in determining the allele of the target enzyme and a reference for comparing the allele of the target enzyme against a reference enzyme known to interact with one or more drugs of interset. The research tool will generally include materials for isolating and/or analyzing the biomolecule from the tumor of the patient, such as a gene chip, labeled or unlabeled probes, monoclonal or polyclonal antibodies, or materials for sequencing the biomolecule or identifying nucleotide polymorphisms. Kits may be developed for specific application in analyzing various different types of tumors and/or drugs, and will generally vary depending upon the type of reference or research material included. The included reference will of course vary depending upon the type of tumor and/or drug under investigation, and will generally include either a reference biomolecule, a reference database or a control as described above. Ancillary reagents may also be included to facilitate the testing.

The below examples are merely intended to illustrate the method of the present invention and should not be construed to limited the scope or the spirit of the invention. This invention is not limited to the preferred embodiments and alternatives heretofore described, to which variations and improvements may be made.

### EXAMPLES

### Example 1

Two subclones of the human mast cell leukemia line HMC1 were used to determine if the wild type and mutant KIT enzymes were able to interact with 2 drug compounds, STI517 and SU9529. Set forth as SEQ ID NO:1 is a nucleotide sequence for the wild type KIT enzyme (amino acid sequence is set forth as SEQ ID NO:2). One subclone, HMC1.1, expressed a valine to glycine substitution in codon 560 (Val560Gly) of the KIT intracellular juxtamembrane region. The other, HMC1.2, expressed the Val560Gly mutation and a second substitution, aspartate to valine in codon 816 (Asp816Val) in the kinase enzymatic pocket. The 2 mutations were originally found together in the HMC1 cell line and were shown to cause SCF-independent constitutive activation of KIT by Furitsu et al., “Identification of mutations in the coding sequence of the proto-oncogene c-KIT in a human mast cell leukemia cell line causing ligand-independent activation of c-KIT product,” J. Clin. Invest., 92:1736-44 (1993). For additional study of wild type and specific mutant forms of KIT, COS cells were used for transient expression of human wild-type and mutated c-KIT complementary DNAs, as described previously by Ma et al., “Inhibition of spontaneous receptor phosphorylation by residues in putatitive α-helix in the KIT intracellular juxtamembrane region,” J. Biol. Chem., 274:13399-13402 (1999). ST1571 is a commercially available drug manufactured by Novartis and developed as an inhibitor of the c-abl gene product. ST1571 has been reported to inhibit wild-type KIT and KIT expressed by HMC1. The other compound, SU9529, is manufactured by SUGEN and has not yet been made commercially available, although it has been published.

Cells were serum-starved overnight, incubated with or without inhibitors for 1 hour and with or without SCF (200 μg/mL, 10 minutes), followed by immunoprecipitation of cell lysates with anti-KIT antibodies (provided by Dr Keith Langley of Amgen, Thousand Oaks, Calif.), fractionation of proteins by sodium dodecyl sulfate-polyacrylamide gel electrophoresis, and immunoblotting with antiphophotyrosine antibody (Upstate Biotechnology, Lake Placid, N.Y.). Blots were stripped and re-probed with anti-KIT antibody as described previously. Ma et al., supra.

Cells cultured in the presence or absence of inhibitors were counted daily in a hemocytometer using trypan blue exclusion. Proliferation assays were repeated at least 3 times. Apoptosis was examined using a DNA fragmentation assay. Briefly, cells were grown in the presence or absence of inhibitors, genomic DNA was isolated and separated by agarose gel electrophoresis, and DNA fragments were visualized under UV light by ethidium bromide staining.

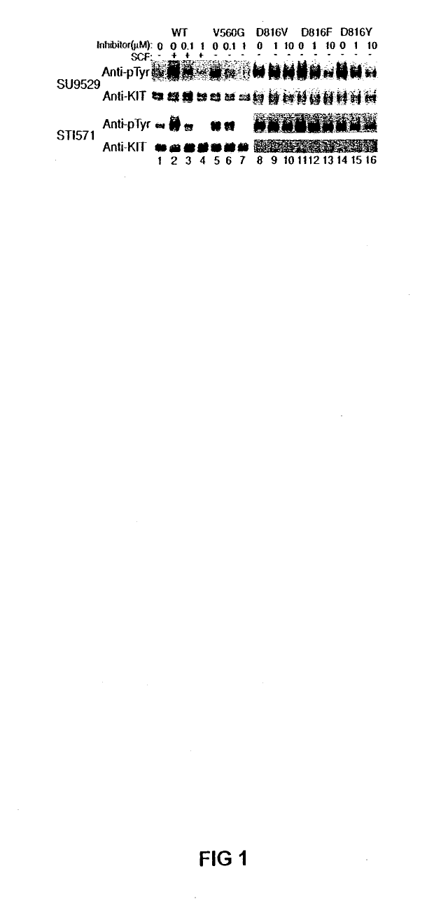

It was discovered that both ST1571 and SU9529 prevent the phosphorylation of wild-type KIT induced by its natural ligand SCF and inhibit SCF-independent constitutive phosphorylation of KIT caused by the Val560Gly juxtamembrane regulatory type (RT) activating mutation at 0.1 to 1 μM (FIG. 1). The antiphosphotyrosine blots of immunoprecipitated KIT expressed in the COS cells exhibited a low level of spontaneous phosphorylation of wild-type (WT) KIT (FIG. 1, lane 1), which increased in response to SCF stimulation (FIG. 1, lane 2). Both inhibitors at 0.1 to 1 μM prevented this ligand-induces phosphorylation (FIG. 1, lanes 3-4). RT mutant KIT with Val560Gly substitution showed a high level of SCF-independent phosphorylation (FIG. 1, lane 5), and the phosphorylation was inhibited by both inhibitors at physiologically achievable (0.1-1 μM) concentrations (FIG. 1, lanes 6-7).

In contrast, both inhibitors failed to inhibit SCF-independent constitutive phosphorylation of KIT containing the Asp816Val enzymatic pocket-type mutation associated with adult human mastocytosis even at 10 μM (FIG. 1). The enzymatic site type (EST) Asp816Val mutation commonly found in human mastocytosis exhibited high spontaneous phosphorylation (FIG. 1, lane 8) but was resistant to inhibition by either KIT inhibitor at 1 to 10 μM (FIG. 1, lanes 9-10). Substitution of phenylalanine or tyrosine for aspartate 816, rarely found in human mastocytosis, also resulted in high spontaneous phosphorylation (FIG. 1, lanes 11 and 14), but these 2 mutant variants responded to the inhibitors at 1 to 10 μM (FIG. 1, lanes 12-13 and 15-16) unlike Asp816Val KIT. However, their response was still an order of magnitude less sensitive than the regulatory type mutant of the wild-type KIT and are not considered valid therapeutic targets with currently available drugs.

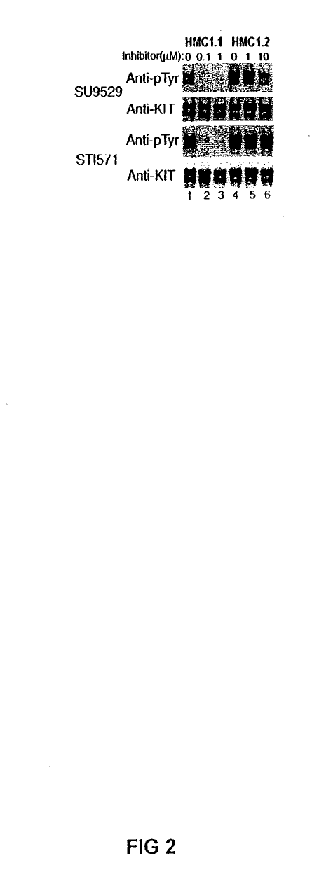

Similarly, both drugs inhibited spontaneous KIT phosphorylation in the HMC 1.1 subclone, which expresses the Val560Gly activating mutation, but at 10 μM they failed to inhibit spontaneous phosphorylation of KIT in the HMC 1.2 subclone, which expresses both the Val560Gly and Asp816Val activating mutations. As illustrated in FIG. 2, antiphosphotyrosine blots of immunoprecipitated KIT expressed in 2 HMC1 subclones showed that spontaneous phosphorylation of KIT containing only the juxtamembrane RT Val560Gly mutation in the HMC 1.1 clone is susceptible to inhibition by both inhibitors at 0.1 to 1 μM (FIG. 2, lanes 1-3). In contrast, spontaneous phosphorylation of KIT with both the Val560Gly the EST Asp816Val mutation in the HMC1.2 clone was resistant to both inhibitors at 1 to 1.0 μM (FIG. 2, lanes 4-6).

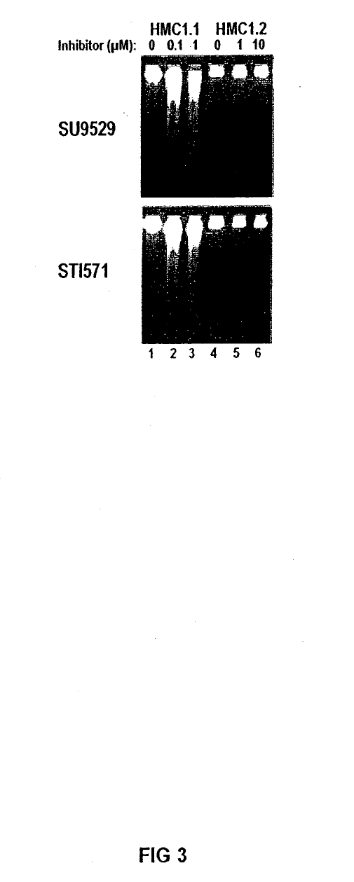

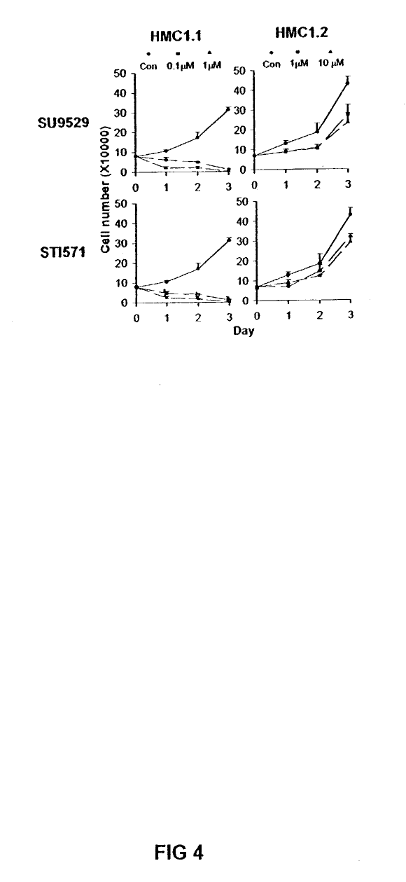

As would be predicted if the activating mutations caused the proliferation of the mast cells and were necessary for their survival, both inhibitors induced apoptosis of the HMC 1.1 cells, causing the death of this line, but failed to kill the HMC1.2 cells (FIGS. 3 and 4). As depicted in FIG. 3, DNA fragmentation assays showed that only the HMC1.1 clone expressing only the Val560Gly juxtamembrane RT mutation underwent apoptosis, as indicated by formation of DNA “ladders” in the presence of the inhibitors at 0.1 to 1 μM (FIG. 3, lanes 2-3). In contrast, the HMC1.2 clone expressing both the Val560Gly mutation and the Asp816Val EST mutation does not exhibit any significant DNA “ladder” in the presences of the inhibitors at 0.1 to 1 μM (FIG. 3, lanes 5-6). As illustrated in FIG. 4, cell proliferation assays showed that incubation of HMC1.1 cells with inhibitors at 0.1 to 1 μM for a 3-day period kills the cells, while treatment of the HMC 1.2 cells with the inhibitors at 1 to 10 μM only slightly inhibits growth of the cells.

Two rare cases of human mastocytosis have been previously described in which other amino acids besides valine are substituted for Asp816 in the enzymatic pocket of the KIT kinase. These 2 variants, involving substitution of either tyrosine or phenylalanine, also cause SCF-independent constitutive phosphorylation of KIT. The 2 mutants, however, were partially inhibited by the KIT inhibitors at 1 to 10 μM (FIG. 1, lanes 11-16), concentrations which are totally ineffective against the most common Asp816Val substituted mutant (FIG. 1, lanes 8-10). Both of these variant enzymatic site type (EST) mutant KITs are an order of magnitude less sensitive than the wild-type or regulatory type (RT) mutant KIT, and neither of these inhibitors appeared to have a high enough therapeutic index to be valid candidates for inhibiting the mutant kinases in a clinical trial. Nevertheless, the data does show that KIT kinases with different residue substitutions in codon 816 of the enzymatic pocket are likely to exhibit differences in susceptibility to specific pharmacologic inhibitors.

The data unequivocally showed that different classes of activating KIT mutations responded differentially to different inhibitors. The data extends previous studies of nonhuman mammalian KIT-activating mutations to the actual mutations found in various forms of human disease and supports the proposals for classification of mutations as either “regulatory type” or “enzymatic site type” mutations, and for classification of human diseases according to the type of the mutations expressed in specific tumors.

Current results also highlight the need to identify specific variants of mutant KIT expressed by individual patients when one contemplates rational therapy. The finding that different amino acid substitutions in codon 816 of the enzymatic pocket represents the first documentation with human mutant activated KIT to support the individualization of drug therapy based on the response of specific mutant proteins to specific drugs. Accordingly, the data suggests that despite the previously reported ability of the KIT inhibitor STI571 to kill an HMC1 line at 1 μM, currently available KIT inhibitors may be ineffective in treating human adult-type mastocytosis. On the other hand, neoplastic processes characterized by RT KIT-activating mutations, such as gastrointestinal stromal tumors, should be susceptible to inhibition by a relatively wide variety of inhibitors, including those that inhibit wild-type KIT.

Different RT mutations, in a given species, show similar sensitivities regardless of the specific amino acid substitution 10 (additional data not shown). This observation supports the concept that the enzymatic pocket in the RT mutants does not differ significantly from the enzymatic pocket of wild-type KIT. It follows that a drug that is a “good fit” for the wild-type enzymatic pocket and is capable of sterically blocking the enzymatic reaction would be likely to be also effective against a RT mutant but would not necessarily be effective against an enzymatic pocket-type mutant. This concept may aid in identifying potentially clinically useful drugs.

The data presented also sheds a unique perspective on the cause of mast cell neoplasms. The fact that the HMC1.1 and 1.2 cell lines are only known to differ by the presence or absence of the Asp816Val mutation makes them an ideal model for determining the role of KIT activation in the factor-independent growth and survival of these cells. The key observation is the fact that both drugs inhibit the RT activating KIT mutation, and both drugs are capable of killing the HMC1.2 cell line, which expresses only that mutation. However, neither of these drugs are capable of inhibiting the enzymatic pocket-type mutation found in the HMC 1.1 clone, and neither are capable of inhibiting the growth and survival of that cell line. Together, these observations show that the ability to kill neoplastic mast cells expressing activated KIT is associated with the ability to inhibit the mutated, activated KIT rather than to inhibit some other unknown kinases.

### Example 2 (Prophetic)

Raf-1 (nucleotide and amino acid sequences are set forth as SEQ ID NO:3 and SEQ ID NO:4, respectively) is a serine-threonine protein kinase that, when activated, phosphorylates and activates mitogen activated protein kinase/extracellular signal related kinase (ERK) kinases (MEK1 and MEK2), stimulating a cascade of intracellular signaling that can influence cell growth and differentiation, and promote neoplastic transformation. Induction of a mutation in codon 361 (G361 S), which causes substitution of the third glycine of the GXGXXG ATP binding motif characteristic of kinase enzymatic sites, results in constitutive activation of MEK and promotes transformation in vitro (Chan et al, “Mutations in conserved regions 1, 2, and 3 of Raf-1 that activate transforming activity,” Molecular Carcinogenesis, 33:189-197 (2002)). Although this mutation has not been described in nature, it is anticipated that its occurrence may contribute to aggressive behavior of a tumor expressing the mutation and would, therefore, represent a potential therapeutic target. However, it is postulated that ATP analogue drugs that bind to the wild type Raf-1 may not also bind to the altered enzymatic site in this enzyme. As a result, such a drug may not be useful as a first line defense against the tumor.

To predict if a particular ATP analogue drug will be effective in treating the tumor of a patient, a biopsy of the tumor is performed. The tumor cells are isolated from the sample and processed in order to recover the cells DNA. The nucleotides encoding the enzymatic site are sequenced and compared to that of a reference Raf-1 enzyme. If the nucleotide sequence of the target Raf-1 enzyme includes a mutation that is known to alter the enzymatic site in a manner that precludes the ATP analogue drug from binding to the enzymatic site, the drug is removed from consideration as a possible candidate for use as a therapeutic drug in that patient. If the nucleotide sequence of the target Raf-1 enzyme includes a mutation that has yet to be defined as inhibitory or permissive with respect to binding of the ATP analogue drug, the target Raf-1 enzyme is subjected to further testing to determine its sensitivity to the ATP analogue drug.

Alternatively, using a labeled probe designed to recognize and bind the Raf-1 protein kinase the Raf-1 target enzyme from the patient is isolated and its enzymatic site sequenced and compared to wild type Raf-1 known to have its activity altered by the ATP analogue drug. If the nucleotide sequence of both enzymes are identical or similar to the extent that there exists no change in the amino acid structure of the enzymatic site, the drug is considered to be a candidate for use as a therapeutic drug for treating the tumor.

Additional sensitivity testing is conducted by causing the expression of the target Raf-1 enzyme by transient transfection and then measuring the sensitivity of the target Raf-1 enzyme to the ATP analogue drug through western blotting. Positive sensitivity results indicate that although the nucleotide sequences of the target Raf-1 enzyme and the wild-type Raf-1 enzyme differ, the mutation does not alter the enzymatic site in a manner such that it inhibits the ability of the ATP analogue drug to alter the activity of the target Raf-1 enzyme. As a result, the ATP analogue drug may be considered to be a candidate for use as a therapeutic drug for treating the tumor.

Example 3 (Prophetic)

Chronic Mylogenic Leukemia (CML) typically arises from a reciprocal translocation between one chromosome 9 and one chromosome 22 of the patient. The translocation results in the modified chromosome 9 being longer than normal and the modified chromosome 22 (referred to as the Philadelphia chromosome) being shorter than normal. The DNA removed from chromosome 9 contains most of the proto-oncogene designated c-ABL, while the break in chromosome 22 occurs in the middle of the gene designated BCR. The resulting Philadelphia chromosome has the 5′ section of BCR fused with most of the c-ABL oncogene. Transcription and translation of the hybrid BCR-ABL gene produces an abnormal (“fusion”) protein that activates constitutively a number of cell activities that normally are turned on only when the cell is stimulated by a growth factor, such as platelet-derived growth factor (PDGF).

The BCR-ABL fusion protein is a tyrosine kinase having its enzymatic site derived from the c-ABL portion. Set forth as SEQ ID NO:5 is the typical nucleotide sequence for the c-ABL portion (amino acid sequence is set forth as SEQ ID NO:6). Mutations to the enzymatic site have been identified, and are believed to be associated with decreased sensitivity to the drug Imatinab. Using the method of the present invention, a preliminary determination can be made as to whether Imatinab, or some other cancer drug, may be effective in treating a patient with chronic leukemia by determining the presence or absence of these mutations.

As described in the examples above, a biopsy of the tumor is performed, with the tumor cells isolated and processed in order to recover their DNA. The nucleotides encoding the enzymatic site are then sequenced and compared to that of a reference BCR-ABL enzyme using typical sequencing techniques or probes (whether by microarray or other method) designed to detect variations in the amino acids or nucleotide sequence associated with the BCR-ABL enzymatic site. If the nucleotide sequence of the target BCR-ABL enzyme includes a mutation that is known to alter the enzymatic site in a manner that precludes the BCR-ABL analogue drug from binding to the enzymatic site (such as mutations to amino acids 244-255 or 311-359 forming the sides of the enzymatic pocket, or amino acids 381-402 forming the enzymatic pocket's activation loop), the drug is removed from consideration as a possible candidate. If the nucleotide sequence includes a mutation that has yet to be defined as inhibitory or permissive with respect to the ability of the BCR-ABL analogue drug to bind, the target BCR-ABL enzyme is subjected to further testing to determine its sensitivity to the analogue drug.

### Example 4 (Prophetic)

The FMS-like tyrosine kinase 3 (FLT3) is a receptor tyrosine kinase that is expressed on early hematopoetic and lymphoid precursor cells, and plays an important role in cell survival and differentiation. Set forth as SEQ ID NO:7 is a nucleotide sequence for the wild-type flt3 gene (amino acid sequence is set forth as SEQ ID NO:8). FLT3 may be activated by mutations called internal tandem duplications (ITDs) that affect the intracellular juxtamembrane of FLT3 (Kiyio et al., “Internal tandem duplication of the FLT3 gene is a novel modality of elongation mutation which causes constitutive activation of the product,” Leukemia 12:1333-37 (1998)), and by point mutations affecting the tyrosine kinase domain, particularly those in codon 835 and 836, which encode amino acids helping to form the enzymatic site of the FLT3 kinase (Yamamoto et al., “Activating mutation of D835 within the activation loop of FLT3 in human hematologic malignancies,” Blood, 97:2434-39 (2001)). FLT3 is expressed by the leukemic cells of most patients with Acute Myeloid Leukemia (AML). FLT3 activating mutations are found in approximately 20% of AML Patients, and their presence is associated with a poorer prognosis (Thiede et al., “Analysis of FLT3-activating mutations in 979 patients with acute myelogenous leukemia: Association with FAB subtypes and identification of subgroups with poor prognosis,” Blood, 99(12):4326-35 (2002)), an observation which suggests that activated FLT3 contributes to disease progression and would be a valid therapeutic target in AML patients.

To predict if a particular drug will be effective in treating a particular FLT3-type AML patient, a sample of the leukemic cells from the patient are collected and processed in order to recover the cells DNA. The DNA is then exposed to a gene chip designed to include individual nucleotide probes representing various polynucleotide sequences found in the enzymatic site of various FLT3 enzymes known to either interact with or to not interact with the particular drug under investigation. Depending upon the resulting binding pattern on the gene chip, the drug is determined to be either a candidate or not a candidate for use as a therapeutic drug for the patient.

The Longley model of activating mutations predicts that drugs that are ATP competitive inhibitors of wild type FLT3 will be effective inhibitors of FLT3 kinase activated by its natural ligand or by regulatory mutations such as those affecting its expression levels or those affecting the intra-molecular regulation of the kinase, typified by the juxtamembrane region ITDs. Inhibitors effective against wild-type FLT3 are, therefore, candidates for treatment of AML in patients whose leukemic cells express FLT3 activated via these mechanisms. However, patients whose leukemic cells express FLT3 activated by mutations affecting the enzymatic site of FLT3, such as those in codons 835 or 836, are more likely to be resistant to such inhibitors, and not a candidate for treatment with the drug absent further testing.

Cells expressing FLT3 activation by mutations affecting the enzymatic site of FLT3 are subjected to additional testing to determine if the altered FLT3 enzymatic site might represent a special therapeutic target found only in leukemic cells. Drug sensitivity testing is conducted by causing the expression of the altered FLT3 enzyme by transient transfection and then measuring the sensitivity of the altered FLT3 enzyme to the drug through western blotting. If the drug only inhibited the mutant enzymatic site and not the wild type enzyme, it may be a candidate for killing leukemic cells with minimal side effects.

I claim:

1. A method for testing the interaction between a drug and an enzyme from a tumor of a patient, the method comprising the steps of:
(a) obtaining from a patient a sample of tumor cells producing an enzyme to be tested for the ability of a drug to interact with the enzyme;
(b) determining the allele of the enzyme by analyzing a biomolecule from the tumor cells, wherein the biomolecule is selected from the group consisting of a nucleotide sequence encoding an enzymatic site of the enzyme, a nucleotide sequence encoding a fragment of the enzymatic site of the enzyme, a polypeptide fragment from the enzymatic site of the enzyme, and the enzyme; and
(c) comparing the allele of the enzyme to a reference to determine the ability of the drug to interact with the enzyme.

2. The method of claim 1 further comprising the step of testing the tumor cells to identify the phosphorylation state of the enzyme.

3. The method of claim 1 further comprising the step of placing the enzyme in direct contact with the drug to further test the sensitivity of the enzyme to the drug.

4. The method of claim 1 wherein the reference is either a reference biomolecule, a reference database, or a control.

5. The method of claim 4 wherein the reference biomolecule is selected from the group consisting of a nucleotide sequence encoding an enzymatic site of a reference enzyme known to interact with the drug, a nucleotide sequence encoding a fragment of the enzymatic site of the reference enzyme, a polypeptide fragment from the enzymatic site of the reference enzyme, the reference enzyme, a polyclonal antibody, a monoclonal antibody and a probe directed to the reference enzyme.

6. The method of claim 5 wherein the enzymatic site comprises a portion of the activation loop for the reference enzyme.

7. The method of claim 4 wherein the reference biomolecule is selected from the group consisting of a nucleotide sequence encoding an enzymatic site of a c-KIT reference enzyme known to interact with the drug, a nucleotide sequence encoding a fragment of the enzymatic site of the c-KIT reference enzyme, a polypeptide fragment from the enzymatic site of the c-KIT reference enzyme, the c-KIT reference enzyme, a polyclonal antibody, a monoclonal antibody and a probe directed to the c-KIT reference enzyme.

8. The method of claim 1 wherein the allele is determined by using either a gene chip, a probe, a monoclonal antibody, a polyclonal antibody, by sequencing the biomolecule, or by performing a nucleotide polymorphism analysis.

9. A method for testing the interaction between a drug and an enzyme from a tumor of a patient, the method comprising the steps of:
(a) obtaining from a patient a sample of tumor cells producing an enzyme to be tested for the ability of a drug to interact with the enzyme;
(b) determining the allele of the enzyme by analyzing a biomolecule from the tumor cells, wherein the biomolecule is selected from the group consisting of a nucleotide sequence encoding an enzymatic site of the enzyme, a nucleotide sequence encoding a fragment of the enzymatic site of the enzyme, a polypeptide fragment from the enzymatic site of the enzyme, and the enzyme; and
(c) comparing the allele of the enzyme to a reference biomolecule to determine the ability of the drug to interact with the enzyme, wherein the reference biomolecule is selected from the group consisting of a nucleotide sequence encoding an enzymatic site of a reference enzyme known to interact with the drug, a nucleotide sequence encoding a fragment of the enzymatic site of the reference enzyme, a polypeptide fragment from the enzymatic site of the reference enzyme, the reference enzyme, a polyclonal antibody, a monoclonal antibody and a probe.

10. The method of claim 9 further comprising the step of testing the tumor cells to identify the phosphorylation state of the enzyme.

11. The method of claim 9 further comprising the step of placing the enzyme in direct contact with the drug to further test the sensitivity of the enzyme to the drug.

12. The method of claim 9 wherein the allele is determined by using either a gene chip, a probe, a monoclonal antibody, a polyclonal antibody, by sequencing the biomolecule, or by performing a nucleotide polymorphism analysis.

13. The method of claim 9 wherein the reference biomolecule is selected from the group consisting of a nucleotide sequence encoding an enzymatic site of a c-KIT reference enzyme known to interact with the drug, a nucleotide sequence encoding a fragment of the enzymatic site of the c-KIT reference enzyme, a polypeptide fragment from the enzymatic site of the c-KIT reference enzyme, the c-KIT reference enzyme, a polyclonal antibody, a monoclonal antibody and a probe directed to the c-KIT reference enzyme.

14. The method of claim 13 wherein the enzymatic site comprises a portion of the activation loop for the reference enzyme.

15. A kit for testing the interaction between a drug and an enzyme from a tumor of a patient, the kit comprising a research tool for determining the allele of the enzyme and a reference for comparison against the allele of the enzyme to determine the ability of the drug to interact with the enzyme.

16. The kit of claim 15 wherein the research tool determines the allele of the enzyme by analyzing a biomolecule from the tumor, wherein the biomolecule is selected from the group consisting of a nucleotide sequence encoding an enzymatic site of the enzyme, a nucleotide sequence encoding a fragment of the enzymatic site of the enzyme, a polypeptide fragment from the enzymatic site of the enzyme, and the enzyme.

17. The kit of claim 15 wherein the research tool is either a gene chip, a probe, a monoclonal antibody, a polyclonal antibody, or materials for sequencing the biomolecule or performing a nucleotide polymorphism analysis.

18. The kit of claim 15 wherein the reference is either a reference biomolecule, a reference database, or a control.

19. The kit of claim 18 wherein the reference biomolecule is selected from the group consisting of a nucleotide sequence encoding an enzymatic site of a reference enzyme known to interact with the drug, a nucleotide sequence encoding a fragment of the enzymatic site of the reference enzyme, a polypeptide fragment from the enzymatic site of the reference enzyme, the reference enzyme, a polyclonal antibody, a monoclonal antibody and a probe.

20. The kit of claim 18 wherein the reference biomolecule is selected from the group consisting of a nucleotide sequence encoding an enzymatic site of a c-KIT reference enzyme known to interact with the drug, a nucleotide sequence encoding a fragment of the enzymatic site of the c-KIT reference enzyme, a polypeptide fragment from the enzymatic site of the c-KIT reference enzyme, the c-KIT reference enzyme, a polyclonal antibody, a monoclonal antibody and a probe directed to the c-KIT reference enzyme.

21. The method of claim 20 wherein the enzymatic site comprises a portion of the activation loop for the reference enzyme.

22. A method for testing the interaction between a drug and a c-KIT target enzyme from a tumor of a patient, the method comprising the steps of:
(a) obtaining from a patient a sample of tumor cells producing a c-KIT target enzyme;
(b) determining the allele of the c-KIT target enzyme by analyzing a biomolecule from the tumor cells, wherein the biomolecule is selected from the group consisting of a nucleotide sequence encoding an enzymatic site of the c-KIT target enzyme, a nucleotide sequence encoding a fragment of the c-KIT target enzymatic site of the enzyme, a polypeptide fragment from the enzymatic site of the c-KIT target enzyme, and the c-KIT target enzyme; and
(c) comparing the allele of the c-KIT target enzyme to a reference to determine the ability of the drug to interact with the c-KIT target enzyme.

23. The method of claim 22 further comprising the step of testing the tumor cells to identify the phosphorylation state of the c-KIT target enzyme.

24. The method of claim 22 further comprising the step of placing the c-KIT target enzyme in direct contact with the drug to further test the sensitivity of the c-KIT target enzyme to the drug.

25. The method of claim 22 wherein the reference is either a reference biomolecule, a reference database, or a control.

26. The method of claim 25 wherein the reference biomolecule is selected from the group consisting of a nucleotide sequence encoding an enzymatic site of a c-KIT reference enzyme known to interact with the drug, a nucleotide sequence encoding a fragment of the enzymatic site of the c-KIT reference enzyme, a polypeptide fragment from the enzymatic site of the c-KIT reference enzyme, the c-KIT reference enzyme, a polyclonal antibody, a monoclonal antibody and a probe directed to the c-KIT reference enzyme.

27. The method of claim 25 wherein the enzymatic site comprises a portion of the activation loop for the reference enzyme.

28. The method of claim 22 wherein the allele is determined by using either a gene chip, a probe, a monoclonal antibody, a polyclonal antibody, by sequencing the biomolecule, or by performing a nucleotide polymorphism analysis.

29. A method for testing the interaction between a drug and a c-KIT target enzyme from a tumor of a patient, the method comprising the steps of:
(a) obtaining from a patient a sample of tumor cells producing a c-KIT target enzyme;
(b) determining the c-KIT target allele of the enzyme by analyzing a biomolecule from the tumor cells, wherein the biomolecule is selected from the group consisting of a nucleotide sequence encoding an enzymatic site of the c-KIT target enzyme, a nucleotide sequence encoding a fragment of the enzymatic site of the c-KIT target enzyme, a polypeptide fragment from the enzymatic site of the c-KIT target enzyme, and the c-KIT target enzyme; and
(c) comparing the allele of the enzyme to a reference biomolecule to determine the ability of the drug to interact with the c-KIT target enzyme, wherein the reference biomolecule is selected from the group consisting of a nucleotide sequence encoding an enzymatic site of a c-KIT reference enzyme known to interact with the drug, a nucleotide sequence encoding a fragment of the enzymatic site of the c-KIT reference enzyme, a polypeptide fragment from the enzymatic site of the c-KIT reference enzyme, the c-KIT reference enzyme, a polyclonal antibody, a monoclonal antibody and a probe directed to the c-KIT reference enzyme.

30. The method of claim 29 further comprising the step of testing the tumor cells to identify the phosphorylation state of the c-KIT target enzyme.

31. The method of claim 29 further comprising the step of placing the c-KIT target enzyme in direct contact with the drug to further test the sensitivity of the c-KIT target enzyme to the drug.

32. The method of claim 29 wherein the allele is determined by using either a gene chip, a probe, a monoclonal antibody, a polyclonal antibody, by sequencing the biomolecule, or by performing a nucleotide polymorphism analysis.
